(19) United States
(12) Patent Application Publication
(10) Pub. No.: US 2026/0330218 A1
(43) Pub. Date: Oct. 22, 2026

(54) COMPACT BIOMARKER DETECTION FOR TRAINING A PREDICTIVE MODEL

(71) Applicant: NEC Laboratories America, Inc., Princeton, NJ (US)

(72) Inventors: Jonathan Warrell, Princeton, NJ (US); Renqiang Min, Princeton, NJ (US)

(21) Appl. No.: 19/466,969

(22) Filed: Feb. 2, 2026

Related U.S. Application Data
(60) Provisional application No. 63/753,009, filed on Feb. 3, 2025.

(51) Int. Cl. G06N 3/08; G06N 3/042; G16B 20/00; G16B 40/30

(52) U.S. Cl. CPC G06N 3/08; G06N 3/042; G16B 20/00; G16B 40/30

(57) ABSTRACT

Systems and methods for detecting biomarkers. The system/method including obtaining a set of proteomics data, clustering the set of proteomics data by percolative clustering using a feature graph, wherein the feature graph has edges between nodes if a pairwise similarity between the nodes satisfies a threshold and the threshold is determined using a minimum cluster size, and clusters in the feature graph are connected components of the feature graph, and selecting a subset of the set of proteomics data as biomarkers for training a predictive model using a loss function of the predictive model and variational optimization with a first variational distribution, subject to a sparsity constraint.

### RELATED APPLICATION INFORMATION

This application claims priority to U.S. Provisional Patent Application No. 63/753,009, filed on Feb. 3, 2025, incorporated herein by reference in its entirety.

### BACKGROUND

### Technical Field

The present invention relates to detecting biomarkers and more particularly detecting compact biomarkers for training a predictive model.

### Description of the Related Art

For clinical utility, biomarkers are often required to use a limited number of proteins or gene measurements. Such biomarkers may include predictors for cancer patient response to immunotherapy using gene expression, or biomarkers to monitor the immune response after TCR therapy or cancer vaccine treatment. Data for learning such biomarkers may make use of whole transcriptome or whole proteome data, but the learned biomarkers should be sparse (for instance, 8-10 genes or proteins or less) so that they are easily monitored clinically. However, in many cases (e.g. clinical trials) the data available for learning such biomarkers is small, so that there may be a large degree of uncertainty in the best set of genes or proteins to use. Aggregating across multiple similar genes often adds stability to such predictors when training using small samples, however, there is a trade-off when selecting a small number of genes between selecting multiple similar genes to add robustness versus choosing genes from multiple pathways to provide maximum information relevant to the patient's condition. Methods such as least absolute shrinkage and selection operator (LASSO), which are typically used to learn sparse biomarkers, do not explicitly control for this trade-off between robustness and informativeness. Therefore, there is a need to learn sparse biomarkers, while allowing for explicit control of this trade-off during the training process.

### SUMMARY

According to an aspect of the present invention, a method is provided for detecting biomarkers for training a predictive model. The method including obtaining a set of proteomics data, clustering the set of proteomics data by percolative clustering using a feature graph, wherein the feature graph has edges between nodes if a pairwise similarity between the nodes satisfies a threshold and the threshold is determined using a minimum cluster size, and clusters in the feature graph are connected components of the feature graph, and selecting a subset of the set of proteomics data as biomarkers for training a predictive model using a loss function of the predictive model and variational optimization with a first variational distribution, subject to a sparsity constraint.

According to another aspect of the present invention, a system is provided for detecting biomarkers for training a predictive model. The system, including a memory device, and one or more processor devices operatively coupled with the memory device to perform operations, the operations including obtain a set of proteomics data, cluster the set of proteomics data by percolative clustering using a feature, wherein the feature graph has edges between nodes if a pairwise similarity between the nodes satisfies a threshold and the threshold is determined using a minimum cluster size, and clusters in the feature graph are connected components of the feature graph, and select a subset of the set of proteomics data as biomarkers for training a predictive model using a loss function of the predictive model and variational optimization with a first variational distribution, subject to a sparsity constraint.

According to another aspect of the present invention, a non-transitory computer program product is provided for detecting biomarkers for training a predictive model. The non-transitory computer program product includes a computer-readable storage medium including a program code, wherein the program code when executed on a computer causes the computer to perform obtain a set of proteomics data, cluster the set of proteomics data by percolative clustering using a feature, wherein the feature graph has edges between nodes if a pairwise similarity between the nodes satisfies a threshold and the threshold is determined using a minimum cluster size, and clusters in the feature graph are connected components of the feature graph, and select a subset of the set of proteomics data as biomarkers for training a predictive model using a loss function of the predictive model and variational optimization with a first variational distribution, subject to a sparsity constraint.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block/flow diagram illustrating a high-level system/method for detecting biomarkers for training a predictive model, in accordance with an embodiment of the present invention;

FIG. 2 is an illustrative example of a practical application of detecting biomarkers for training a predictive model, in accordance with an embodiment of the present invention;

FIG. 3 is a block/flow diagram illustrating a system/method for detecting biomarkers for training a predictive model, in accordance with an embodiment of the present invention;

FIG. 4 is a flow diagram illustrating a system/method for detecting biomarkers for training a predictive model, in accordance with an embodiment of the present invention; and

FIG. 5 is a block/flow diagram illustrating a system for video detecting biomarkers for training a predictive model, in accordance with an embodiment of the present invention.

### DETAILED DESCRIPTION OF PREFERRED EMBODIMENTS

In accordance with embodiments of the present invention, systems and methods are provided for detecting biomarkers for training a predictive model.

The system/method relates to selecting a subset of features where there is a large number of features and it is expected that features which may be noisy individually will form clusters that may reinforce each other. Examples include genomics and neuroscience data, causal analysis, and model compression. The system/method clusters the features through percolative clustering using a feature graph. Then the system/method selects the lowest amount of features for a subset of features as biomarkers while still having predictive power. The system/method can select the subset of features using a loss function of a predictive model and variational optimization.

An improvement in accordance with embodiments of the present invention includes the ability to identify sparse models. This is an improvement because sparse models explicitly balance robustness and informativeness of features in the features selected by the model. For example, if too many features are selected, one may have a lot of correlation, but not much predictive power when generalizing to new data. Too few features and one may lose predictive power. That's why there is a need to balance the two opposing needs.

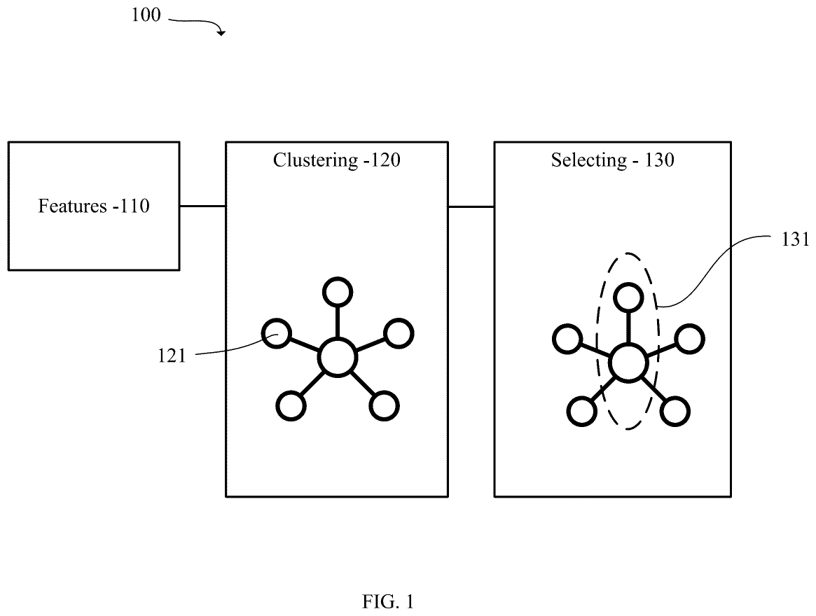

Referring now in detail to the figures in which like numerals represent the same or similar elements and initially to FIG. 1, a high-level system/method 100 for biomarker detection is illustratively depicted in accordance with one embodiment of the present invention. The system/method 100 receives a set of features 110. The features 110 can relate to biomarkers such as proteins or proteomics data. The system/method 100 then clusters 120 the features 110 using percolative clustering using a feature graph 121. Then, the system/method 100 selects 130 the subset of features 131 as biomarkers for training a predictive model. The selection 130 can be done using variational optimization and a loss function of the predictive model while subject to a sparsity constraint.

In an embodiment, the predictive model can be a graph neural network (GNN). Continuing with the embodiment, the GNN uses a graph structure in which edges are permitted only between features assigned to a same cluster.

In an embodiment, the predictive model can be a logical regression model. Continuing with this embodiment, the logical regression model's loss function can include cross-entropy loss. Additionally, the loss function can include ridge-regression, least absolute shrinkage and selection operator (LASSO), or elastic net penalties.

A practical application of embodiments of the invention can include creation of a treatment plan.

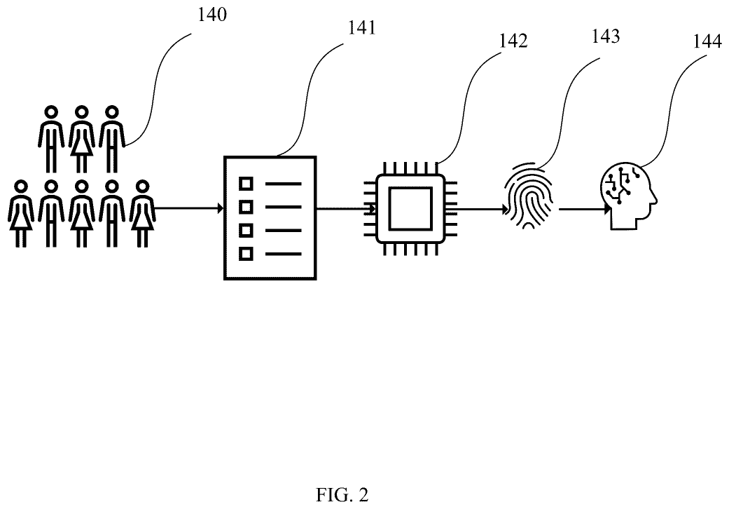

Referring to FIG. 2, in an embodiment, a set of proteomics data 141 from patients 140 is input into a computer 142 and biomarkers 143 are determined using the present embodiment by percolatively clustering the set of proteomics data 141 and selecting a subset of the set of proteomics as biomarkers 143. The biomarkers 143 are then used to train a predictive model 144 which can come up with a treatment plan related to the biomarkers 143. For example, the treatment plan from the predictive model 144 can be a personalized cancer vaccine treatment plan.

Another practical application of embodiments of the invention includes training the predictive model with the detected biomarkers. However, it should be noted that training the predictive model is not limited to using only the detected biomarkers but can include using other features and datasets too.

Another practical application of embodiments of the invention includes discovering links between diseases and biomarkers. For example, the predictive model trained with the biomarkers can predict which hereditary diseases can be connected to the biomarkers.

The system/method 100 can obtain a cohort/dataset of patients with genomic measurements, x₁ . . . x_{D}, where xᵢ is a vector of genomic measurements (e.g. protein levels) associated with patient i, and D is the size of the dataset. Each vector is of length P, where P is the number of features. Additionally, the system/method 100 can predict a response variable, e.g. response/resistance to a treatment from these measurements, which the system/method denotes y₁ . . . y_{D}. For convenience, below the system/method 100 assumes xᵢ∈ [symbol] and yᵢ∈{0,1}, although the system/method 100 may be used with regression problems on arbitrary input and output spaces. A goal is to learn a sparse biomarker, such that the total number of input features T is less than a maximum Tₘₐₓ≤P. Hence, the system/method 100 searches for a subset of features S, with |S|=T≤Tₘₐₓ, and a function, φ: [symbol] →{0,1}, where φ may belong to an arbitrary predefined model class. For convenience, the system/method 100 can consider that φ is either a Graph Neural Network (GNN), or a logistic regression (LR) model. A goal of the system/method 100 is to find a function φ that achieves good generalization on unseen data.

The system/method 100 can provide a means of finding such functions φ. The system/method can have a means of clustering the features in the dataset, so that a cluster index C(p)∈{1 . . . K} is assigned to each feature p. The system/method 100 then attempts to find the optimal predictor @ on the training data, such that M predictors are selected from at least N clusters (hence, NM≤T). The underlying optimization problem is thus:

$${min}_{ϕ , S} {\sum }_{i} ℒ ( ϕ ( {x}_{i , S} ) , {y}_{i} )$$

$$s . t . \vert S \vert \leq {T}_{max} , \vert \{ k ; \vert {S}_{k} \vert \geq N \} \vert \geq M \qquad ( 1 )$$

where Sₖ={p;p∈S∧C(p)=k}, L is a loss function (which is assumed for convenience to be non-negative and bounded), and the system/method write x_{i,S} for the restriction of xᵢ to features in the selected subset S⊂{1 . . . P}.

The system/method 100 is a specific implementation designed to perform biomarker discovery by optimizing the above objective, using percolation clustering to define C(p), and Variational Optimization to search for a solution of Eq. 1. Specifically, the system/method 100 uses a clustering procedure motivated by the analysis of percolating systems in statistical mechanics to find a pseudo-‘critical region’ of parameter space to select feature clusters.

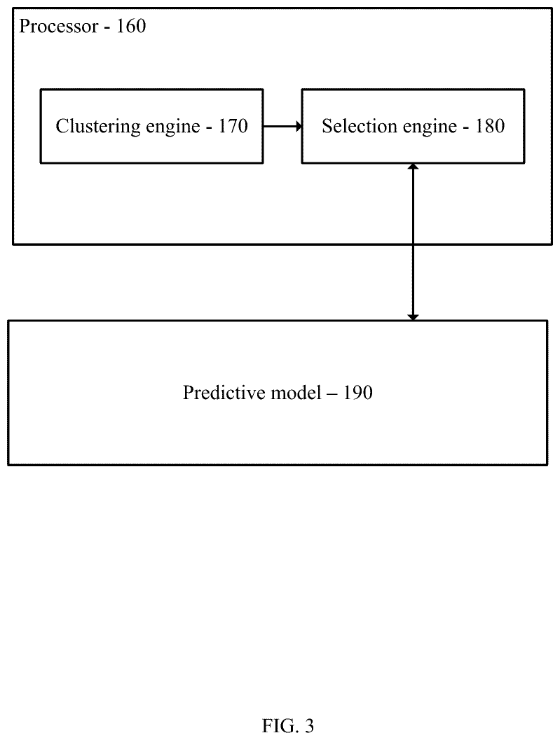

Referring to FIG. 3, a processor 160 is executing a clustering engine 170 and a selection engine 180. The clustering engine 170 clusters the features and the selection engine 180 selects the subset of features as biomarkers. The selection engine 180 can use the loss function of the predictive model 190 to select the subset of features as biomarkers. Then the selection engine 180 sends the biomarkers to the predictive model 190 where the predictive model is trained using the biomarkers 190.

The clustering engine 170 clusters the features in the dataset with percolatively clustering using a features graph. The clustering engine 170 uses a clustering procedure which analogously finds a pseudo ‘critical region’ of parameter space to select feature clusters. Therefore, the clustering engine 170 calculates the Pearson Correlation Coefficient between each pair of features in our training data, and adds an edge between them if this value is above a threshold τ:

E(τ)={(p₁,p₂)∈{1 . . . P}²;corr([x_{1,p}_{₁}, . . . ,x_{D,p}_{₁}],[x_{1,p}_{₂}, . . . ,x_{D,p}_{₂}])≥τ} (2)
where corr(v, w) is the Pearson Correlation Coefficient between vectors v and w. Additionally, the clustering engine 170 defines the associated clustering function as:

$${C}_{τ} ( p ) = {min}_{q} \{ {p}^{′} ; p ∈ CC ( q , E ( τ ) ) \} \qquad ( 3 )$$

where CC(q, E(τ)) is the connected component of the graph defined by the edge relation E(τ) including node q. The clustering engine 170 chooses t by fixing a minimum cluster size of interest, Kₘᵢₙ, and maximizing the number of clusters whose size is at least Kₘᵢₙ:

$${τ}^{*} = {max}_{τ} {\sum }_{k} [ \vert \{ p ; {C}_{τ} ( p ) = k \} \vert \geq {K}_{min} ] . \qquad ( 4 )$$

After the clustering engine 170 clusters the features, the selection engine 180 then selects a subset of features for a predictive model. The subset of features can include biomarkers.

Eq. 1 involves optimization over both continuous and discrete variables. For this reason, the selection engine 180 uses a discrete optimization approach based on Variational Optimization. The selection engine 180 assumes, given a subset of features S, the selection engine 180 can easily find the optimal function φ_{S} associated with this subset, for instance by gradient descent. The selection engine 180 therefore introduces an auxiliary variable, θ∈ [symbol] , and let:

S_{θ}={p;θₚ>0}

The optimization problem in Eq. 1 is thus equivalent to a minimization problem over φ and θ, where the selection engine 180 replaces all instances of S with S_{θ}, where S_{θ} corresponds to set of features generated by thresholding θₚ at zero.

The selection engine 180 then introduces the following variational distribution over θ:

Q_{μ,σ}(θ)= [symbol] (θ;μ,σ)
where μ is a vector of length P, σ is a scalar, and [symbol] (θ;μ,σ) is a normal distribution with mean μ and standard deviation σ. The selection engine 180 further introduces the following variational objective function:

$$F ( θ ) = \{ \begin{matrix}( \frac{1}{D} ) {\sum }_{T} ℒ ( {ϕ}_{{S}_{θ}}^{⋆} ( {x}_{i , {S}_{θ}} ) , {y}_{i} ) − 0 , if \vert {S}_{θ} \vert \leq {T}_{max} ⋀ \vert \{ k ; \vert {S}_{θ , k} \vert \geq N \} \vert \geq M \\ 0 , otherwise\end{matrix}$$

$$where {ϕ}_{s}^{*}$$

denotes the optimal function φ trained on features p∈S, and O is a constant offset,

$$O = {max}_{{s}^{′} , y} L ( {ϕ}_{{s}^{′}}^{*} ( {x}_{{s}^{′}} ) , y ) ,$$

which ensures F(θ) is non-negative. Then, by definition, assuming F(θ) is not the constant 0 function, for any solution

$${θ}^{*} ∈ {arg max}_{θ} F ( θ ) , ( {ϕ}_{{s}^{∘}}^{*} , {S}_{{b}^{⋆}} )$$

is a solution to Eq. 1. Further, the variational bound can be defined as:

$${max}_{μ , σ} {𝔼}_{θ ∼ {0}_{μ , r}} [ F ( θ ) ] \leq {max}_{θ} F ( θ )$$

and the selection engine 180 may thus optimize Eq. 1 by performing variational optimization over θ, for instance by using the smoothing-based optimization updates:

$${μ}_{t + 1} = \frac{{\sum }_{s} {f}_{s}^{t} {θ}_{s}^{t}}{{\sum }_{s} {f}_{s}^{t}}$$

$${σ}_{t + 1} = \sqrt{\frac{{\sum }_{s} {f}_{s}^{t} {\vert {θ}_{s}^{t} - {μ}_{t} \vert}_{2}^{2}}{P {\sum }_{s} {f}_{s}^{t}}}$$

$$where {θ}_{s}^{t}$$

for s∈{1 . . . S} are samples drawn from Qₜ= [symbol] (−;μₜ,σₜ), and

$${f}_{x}^{t} = F ( {θ}_{x}^{t} ) .$$

The updates in Eq. 9 are guaranteed to increase [symbol] [F(θ)] in expectation.

While the above describes a Variational Optimization approach to sparse feature selection for fixed M, N and Kₘᵢₙ, an additional variational distribution Q′ may be placed over these parameters and optimized in an outer-loop (where M, N and Kₘᵢₙ represent respectively the number of features per group, the number of groups and the minimum cluster size of interest, as described above).

To prevent this distribution collapsing on a solution with singleton clusters and M=1, the performance on a validation partition should be used as the score over which to optimize Q′ (i.e. the goal is to find the setting of M, N and Kₘᵢₙ that achieves the best generalization performance).

As noted, the above approach may be used for the case that φ is an arbitrary kind of prediction model. Below, the system/method can use implementations where φ is a GNN and a LR model.

For the GNN case, the system/method may use the clustering function C(p) to constrain the graph structure of the GNN. Hence, for a given set of selected features S, the graph structure for the GNN is defined as E(S)={(p₁,p₂);p₁∈S∧p₂∈S∧C(p₁)=C(p₂)}. Such a graph structure promotes sharing between features that are expected to belong to similar biological pathways (for instance).

For the LR case, φ may be learned by simply using the cross-entropy loss; alternatively, ridge-regression, LASSO or elastic net penalties may be incorporated into the loss function [symbol] .

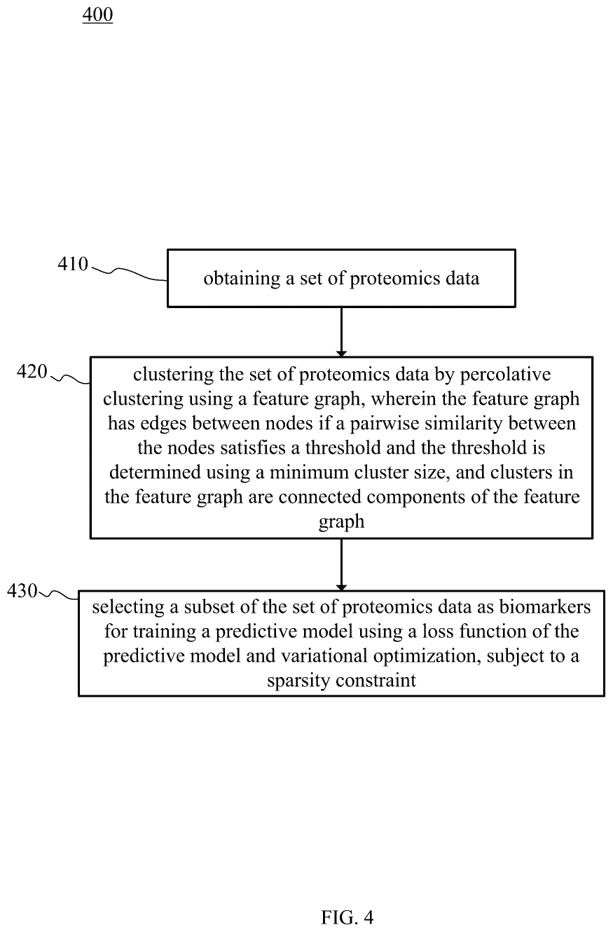

Referring now to FIG. 4, a flow diagram 400 of a high-level overview of biomarker detection, in accordance with an embodiment of the present invention.

In block 410, a set of proteomics data is obtained. The dataset can be representative of patients with genomic measurements.

In block 420, the set of proteomics data are clustered using percolative clustering using a feature graph. The feature graph can have edges between nodes if the pairwise similarity between the nodes satisfies a threshold. The threshold can be determined using a minimum cluster size. The clusters in the feature graph can be connected components of the feature graph.

In block 430, a subset of the set of proteomics data is selected for training a predictive model using a loss function of the predictive model and variational optimization, subject to a sparsity constraint. The selected subset of features can include biomarkers for training a predictive model. The predictive model can be a GNN or a logical regression model. If the predictive model is a GNN, then the GNN can use a graph structure in which edges are permitted only between features assigned to a same cluster. If the predictive model is a logical regression model, then the loss function can be cross-entropy loss.

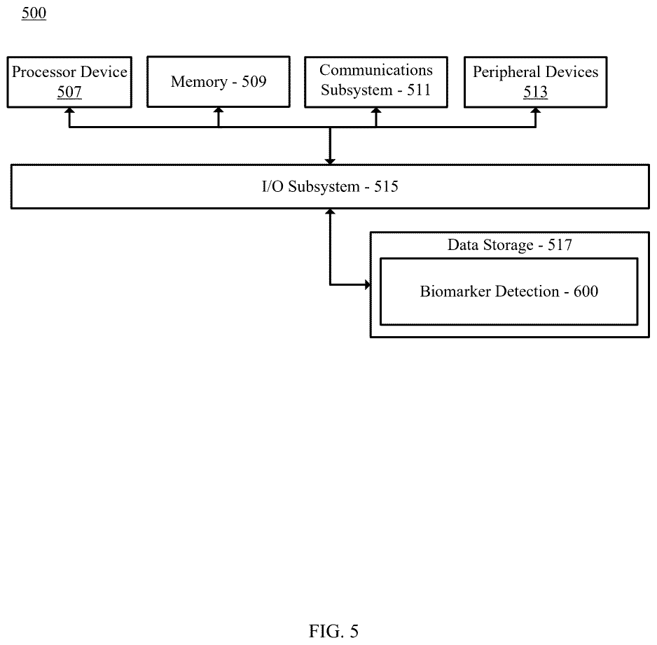

Referring now to FIG. 5, a block diagram of a computer system for biomarker detection, in accordance with an embodiment of the present invention.

The computing device 500 illustratively includes the processor device 507, the input/output (I/O) subsystem 515, the memory 509, the data storage device 517, and the communications subsystem 511, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 509, or portions thereof, may be incorporated in the processor device 507 in some embodiments.

The processor device 507 may be embodied as any type of processor capable of performing the functions described herein. The processor device 507 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 509 may be embodied as any type of volatile or non-volatile memory or data storage capable of performing the functions described herein. In operation, the memory 509 may store various data and software employed during operation of the computing device 500, such as operating systems, applications, programs, libraries, and drivers. The memory 509 is communicatively coupled to the processor device 507 via the I/O subsystem 515, which may be embodied as circuitry and/or components to facilitate input/output operations with the processor device 507, the memory 509, and other components of the computing device 500. For example, the I/O subsystem 515 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 515 may form a portion of a system-on-a-chip (SOC) and be incorporated, along with the processor device 507, the memory 509, and other components of the computing device 500, on a single integrated circuit chip.

The data storage device 517 may be embodied as any type of device or devices configured for short-term or long-term storage of data such as, for example, memory devices and circuits, memory cards, hard disk drives, solid state drives, or other data storage devices. The data storage device 517 can store program code for biomarker detection 600. Any or all of these program code blocks may be included in a given computing system.

The communications subsystem 511 of the computing device 500 may be embodied as any network interface controller or other communication circuit, device, or collection thereof, capable of enabling communications between the computing device 500 and other remote devices over a network. The communications subsystem 511 may be configured to employ any one or more communication technology (e.g., wired or wireless communications) and associated protocols (e.g., Ethernet, InfiniBand®, Bluetooth®, Wi-Fi®, WiMAX, etc.) to affect such communication.

As shown, the computing device 500 may also include one or more peripheral devices 513. The peripheral devices 513 may include any number of additional input/output devices, interface devices, and/or other peripheral devices. For example, in some embodiments, the peripheral devices 513 may include a display, touch screen, graphics circuitry, keyboard, mouse, speaker system, microphone, network interface, and/or other input/output devices, interface devices, global positioning system (GPS), camera, and/or other peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be employed. Moreover, additional processors, controllers, memories, and so forth, in various configurations can also be utilized. These and other variations of the computing device 500 are readily contemplated by one of ordinary skill in the art given the teachings of the present invention provided herein.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A method for detecting biomarkers for training a predictive model, the method comprising:
obtaining a set of proteomics data;
clustering the set of proteomics data by percolative clustering using a feature graph, wherein the feature graph has edges between nodes if a pairwise similarity between the nodes satisfies a threshold and the threshold is determined using a minimum cluster size, and clusters in the feature graph are connected components of the feature graph; and
selecting a subset of the set of proteomics data as biomarkers for training a predictive model using a loss function of the predictive model and variational optimization with a first variational distribution, subject to a sparsity constraint.

2. The method of claim 1, wherein the predictive model is a graph neural network (GNN).

3. The method of claim 2, wherein the GNN uses a graph structure in which edges are permitted only between features assigned to a same cluster.

4. The method of claim 1, the method further comprising:
training the predictive model using the biomarkers.

5. The method of claim 1, wherein the predictive model is a logical regression model.

6. The method of claim 5, wherein the loss function is a cross-entropy loss.

7. The method of claim 1, wherein the selecting is done using a second variational distribution.

8. The method of claim 7, wherein a performance on a validation partition is used as a score to optimize the additional variational distribution.

9. A system, comprising:
a memory device; and
one or more processor devices operatively coupled with the memory device to perform operations, the operations including:
obtain a set of proteomics data;
cluster the set of proteomics data by percolative clustering using a feature, wherein the feature graph has edges between nodes if a pairwise similarity between the nodes satisfies a threshold and the threshold is determined using a minimum cluster size, and clusters in the feature graph are connected components of the feature graph; and
select a subset of the set of proteomics data as biomarkers for training a predictive model using a loss function of the predictive model and variational optimization with a first variational distribution, subject to a sparsity constraint.

10. The system of claim 9, wherein the predictive model is a graph neural network (GNN).

11. The system of claim 10, wherein the GNN uses a graph structure in which edges are permitted only between features assigned to a same cluster.

12. The system of claim 9, wherein the feature graph has edges between nodes if a pairwise similarity between the nodes satisfies a threshold.

13. The system of claim 12, wherein the threshold is determined using a minimum cluster size.

14. The system of claim 9, wherein clusters in the feature graph are connected components of the feature graph.

15. The system of claim 9, the operations further including:
training the predictive model using the biomarkers.

16. The system of claim 9, wherein the predictive model is a logical regression model.

17. A non-transitory computer program product comprising a computer-readable storage medium including a program code, wherein the program code when executed on a computer causes the computer to perform:
obtain a set of proteomics data;
cluster the proteomics data by percolative clustering using a feature graph, wherein the feature graph has edges between nodes if a pairwise similarity between the nodes satisfies a threshold and the threshold is determined using a minimum cluster size, and clusters in the feature graph are connected components of the feature graph; and
select a subset of the set of proteomics data as biomarkers for training a predictive model using a loss function of the predictive model and variational optimization with a first variational distribution, subject to a sparsity constraint.

18. The non-transitory computer program product of claim 17, wherein the predictive model is a graph neural network (GNN).

19. The non-transitory computer program product of claim 18, wherein the GNN uses a graph structure in which edges are permitted only between features assigned to a same cluster.

20. The non-transitory computer program product of claim 17, the program code further including:
training the predictive model using the subset of features.
